(19) United States
(12) Patent Application Publication
(10) Pub. No.: US 2026/0367055 A1
(43) Pub. Date: Nov. 19, 2026

(54) EMISSIONS DETERMINATION SYSTEM AND PROCESS

(71) Applicant: Schlumberger Technology Corporation, Sugar Land, TX (US)

(72) Inventors: Malik Alkhadra, Dhahran (SA); Yermek Kaipov, Dhahran (SA)

(21) Appl. No.: 19/209,569

(22) Filed: May 15, 2025

(51) Int. Cl. G01N 33/00; G01N 9/36

(52) U.S. Cl. CPC G01N 33/0062; G01N 9/36

(57) ABSTRACT

Embodiments of the invention provide for methods and systems for determining gas emissions. In one embodiment, a method for continuous determining of gas emissions includes providing a gas feed to a gas flaring device, continuously analyzing a gas composition of the gas feed with an analyzer, providing gas composition data of the gas feed from the analyzer to a processor, providing a gas volumetric rate or a gas mass rate to the processor, providing a gas flaring device combustion efficiency to the processor and determining, by the processor, the gas flaring device emissions. A system utilizing embodiments of the methods is also provided.

### BACKGROUND

### Field of the Disclosure

The present disclosure is related to processes and systems for determining gas emissions, in particular processes and systems for determining flare gas emissions.

### Background

In the oil and gas industry, gas flaring using gas flares, or flare burners, remains an economical approach for handling waste fluids produced in exploration wells or during well testing operations. Unburned soot and gaseous emissions are two kinds of environmental pollution linked to gas flaring operations and are regulated by various environmental agencies. Measurement of such pollution can be used for compliance with environmental regulations and permits and ensure accurate reporting to the necessary environmental agencies.

Currently systems use manual inputs, estimates, and are periodically taken. This can lead to inconsistent data and estimates and potentially fail to be in compliance with the necessary regulations and permits.

Accordingly, there is a need for process and system, for accurately determining emissions from gas flares.

### SUMMARY

A summary of certain embodiments described herein is set forth below. It should be noted that these aspects are presented merely to provide the reader with a brief summary of these certain embodiments and that these aspects are not intended to limit the scope of this disclosure.

Aspects of the present disclosure provide systems and methods for determining gas emissions.

In one embodiment, a method for continuous determining of gas emissions is provided. A system utilizing embodiments of the methods is also provided. A non-transitory computer readable storage medium comprising instructions which, when executed, cause a machine to perform the method described herein is also provided.

In one aspect, a method for continuous determining of gas emissions is provided. The method for continuous determining of gas emissions includes providing a gas feed to a gas flaring device, continuously analyzing a gas composition of the gas feed with an analyzer, providing gas composition data of the gas feed from the analyzer to a processor, providing a gas volumetric rate or a gas mass rate to the processor, providing a gas flaring device combustion efficiency to the processor, and determining, by the processor, the gas flaring device emissions.

In another aspect, a method for determining gas emissions is provided. The method for determining gas emissions includes providing a continuous feed of a gas to a gas flaring device, continuously analyzing the gas composition of the continuous feed of the gas, providing continuous data to processor, including providing gas composition data to the processor, providing a gas volumetric rate or a gas mass rate to the processor, providing a gas flaring device combustion efficiency to the processor and determining, by the processor, the gas flaring device emissions.

In another aspect, a system for continuous determining of gas emissions is provided. The system for continuous determining of gas emissions includes a gas flaring device, a gas feed line fluidly coupled to the gas flaring device, a continuous gas analyzer coupled to the gas feed line, a processor in electronic communication with the continuous gas analyzer, wherein the processor is adapted to receive: gas composition data from the continuous gas analyzer, gas volumetric rate or gas mass rate data, and gas flaring device combustion efficiency data, and to output emission data, and a display device adapted to receive continuous output emission data from the processor.

In another aspect, a non-transitory computer readable storage medium is provided. The non-transitory computer readable storage medium includes instructions which, when executed, cause an apparatus, a machine, a processor, or a system to at least obtain: continuous gas composition data of a gas feed coupled to a gas flaring device, continuous gas volumetric rate or a gas mass rate of the gas feed, and gas flaring device combustion efficiency, calculate continuously gas flaring device emissions of the gas feed, and provide data of the gas flaring device emissions to an output device.

### BRIEF DESCRIPTION OF DRAWINGS

The appended figures illustrate only exemplary embodiments and are therefore not to be considered limiting of the scope of the disclosure, as the disclosure may admit to other equally effective embodiments.

FIG. 1 is one embodiment of a system to determine gas emissions from a gas flare;

FIG. 2 is one embodiment of a block diagram of a system design;

FIG. 3 is one embodiment of a block diagram of an example implementation of the example system of FIG. 1.

FIG. 4 is one embodiment of a chart from the output of the processor illustrating emissions data output; and

FIG. 5 is one embodiment of a chart from the output of the processor illustrating gas concentration input data.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Aspects of the present disclosure provide systems and methods for determining gas emissions.

In the following description, numerous details are set forth to provide an understanding of some embodiments of the present disclosure. It is to be understood that the following disclosure provides many different aspects, or examples, for implementing different features of various embodiments. Specific examples of components and arrangements are described below to simplify the disclosure. These are, of course, merely examples and are not intended to be limiting. However, it will be understood by those of ordinary skill in the art that the system and/or methodology may be practiced without these details and that numerous variations or modifications from the described aspects are possible. This description is not to be taken in a limiting sense but rather made merely for the purpose of describing general principles of the implementations. The scope of the implementations described should be ascertained with reference to the issued claims.

Oil and gas drilling is a complex process involving the penetration of subsurface formations to extract hydrocarbons from reservoirs. The process is typically carried out using a drilling rig that creates a wellbore, through which the hydrocarbons can be brought to the surface. Excess or unwanted hydrocarbon gases and/or liquids extracted from the well may be processed by burning in a gas flare, or flare burner, resulting in gas emissions and soot.

It is believed that the systems and processes described herein provide for comprehensive and accurate methods for estimating gas emissions during gas flaring operations in real-time by integrating live gas composition analysis with mathematical calculations. The systems and processes described herein capture and display real-time data on gas emissions through software, which provides an ongoing summation of emissions from gas flaring in terms of pollutant gases, carbon dioxide equivalents, and soot, measured in metric tonnes.

It is further believed that the systems and processes described herein allow for precise monitoring and evaluation of the environmental impact of gas flaring, facilitating better management of gas emissions and compliance with regulatory standards.

In one embodiment, the systems and processes are designed to measure and analyze gas emissions released into the atmosphere in real-time during flowback operations.

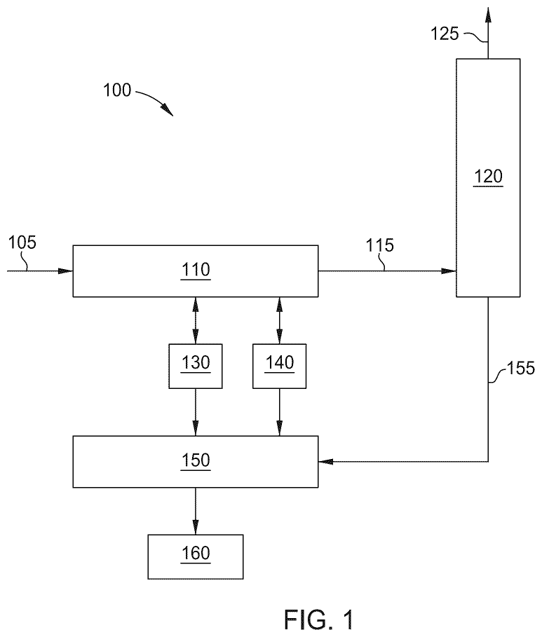

FIG. 1 depicts an example system 100 for determining gas emissions according to one embodiment of the invention. It should be understood that FIG. 1 merely depicts an example system, and that the processes described herein may be performed in any suitable system.

FIG. 1 shows a schematic diagram of a system 100 for continuous determining of gas emissions. The system includes a gas line 110 fluidly coupled 115 to a gas flare 120. The gas flare has processed the gas feed and releases emissions 125. The gas line 110 is in fluid communication with a gas source 105. A gas analyzer 130 is coupled to or integrated into, and adapted to measure the gas composition of the gas line 110. The gas analyzer 130 may be an in-line gas analyzer or an in-situ gas analyzer. Alternatively, gas analyzer 130 may be a separate device or mobile device that can be disposed on the gas line 100 to analyze the gas feed for periods of time before removal. A flow rate meter 140 is coupled to and adapted to measure the flow rate as a gas volumetric flow rate or a mass flow rate of the gas line 110.

The gas analyzer 130 and flow meter 140 are in continuous electronic communication, and provide continuous data thereto, a processer 150. Processor 150 may also be referred to as a calculator. The processor 150 is adapted to receive input data from the gas analyzer 130 and flow meter 140, and further includes mathematical models, formulas, and data processing algorithms, often trade secret in nature, to determine gas emissions from the input data. Processor 150 is in electronic communication with one or more output devices 160 and provides continuous input data and output data to such one or more output devices 160. The input and output data may also be provided to other electronic devices as needed, such databases, data storage devices, and plant monitoring systems.

The gas line 110 can be any gas line adapted to carry hydrocarbon gas and/or liquids from a source 105. An example of a gas line is a supply gas lines from a separator at a well site, a waste line from plant operations, a flowback operation line, or a fuel line.

The gas flare 120 is a type commonly used in the oil and gas industry. The gas flare 120, or gas burner or flare burner, may be any gas flare equipment, such as multiphase flare equipment, for combustion of fluid flows including gas condensate, natural gas, oil, and/or water of any type. The gas flare 120 may be of any type, such as such an open-atmosphere or open-air gas flare. Alternatively, any other configuration and/or type of gas flare burner, liquid flare burner, or multiphase flare burner can be used.

The gas analyzer 130 employs in-line gas analysis technologies and/or in-situ gas analysis technologies to determine and record the gas composition in real-time. Suitable gas analysis technologies include, and are not limited to, Raman spectroscopy, tunable filter spectroscopy, mass spectroscopy, gas chromatography, tunable diode laser absorption spectroscopy, Fourier transform infrared spectroscopy, cavity ring-down spectroscopy, or a combination thereof. Preferred technologies include Raman spectroscopy, tunable filter spectroscopy, and gas chromatography, among others. One or more gas analyzers may be used as the gas analyzer 130. A preferred gas analyzer is based on Raman Spectroscopy technology.

The gas analyzer 130 is adapted to measure one or more of the following components selected from the group consisting of methane, ethane, propane, isobutane, n-butane), isopentane, n-pentane, nitrogen gas (N₂), carbon dioxide, hydrogen sulfide, and combinations thereof. In one embodiment, the gas composition of the various components is expressed in weight and molar mass percentages.

The specific gravity of the gas feed may be calculated by taking the weighted average of the molar weights of each gas component, based on the percentages in the gas mixture by multiplying the molar weight of each component by the component's percentage, summing the values for all the components, and then dividing by the molar weight of air. This calculation can be performed concurrently with the continuous measurements of the gas composition by the gas analyzer 130. The gas volumetric rate measurement obtained from the flow meter 140 can use the calculated gas specific gravity in real-time instead of manual input.

The flow meter 140 employs one or more in-line gas metering devices or in-situ gas metering devices to measure the gas volumetric rate (MMscf/d) or gas mass rate (kg/s), or both, in real-time. Suitable gas metering devices may include, and are not limited to, devices selected from the group consisting of orifice metering devices, Coriolis metering devices, diaphragm metering devices, rotary metering devices, turbine metering devices, and combinations thereof.

Processor 150 comprises a mathematical model that enables calculations of the emissions released into the atmosphere in real time based on the continuously provided gas composition data and volumetric or mass flowrates combined with an inputted combustion efficiency. The processor may also be referred to as the Dynamic Emission Calculator. The calculated emissions are expressed in metric tonnes and include CO₂ (carbon dioxide), CH₄ (methane), NOx (nitrogen oxides), CO (carbon monoxide), SO₂ (sulfur dioxide), and soot. The calculated emissions for CO₂, CO, CH₄, and soot are used to calculate the CO₂ equivalents of the emission gases, and the CO₂ equivalents are measured in metric tonnes.

The combustion efficiency data may be provided in two manners. The combustion efficiency data may be provided as a preset value based on a measured combustion efficiency of the gas flare. The combustion efficiency data may be provided at 98%, in line with the “EPA Air Emissions Factors and Quantification, AP-42: Compilation of Air Emissions Factors, Appendix A: Miscellaneous Data and Conversion Factors.” Alternatively, the combustion efficiency 155 may be provided in real-time from systems and processes monitoring and adjusting combustion efficiency of the gas flare.

In certain embodiments, the processor 150 may include a microprocessor, a microcontroller, a processor module or subsystem, a programmable integrated circuit, a programmable gate array, a digital signal processor (DSP), or another control or computing device. Alternatively, or additionally, the processor 150 may include discrete electronic components coupled to a printed circuit board, integrated circuitry (for example, Application Specific Integrated Circuits (ASIC)), and/or programmable logic devices (for example, a Field Programmable Gate Arrays (FPGA)).

In certain embodiments, one or more data models may be implemented as computer program logic for use with the processor 150. The computer program logic may be embodied in various forms, including a source code form or a computer executable form. Source code may include a series of computer program instructions in a variety of programming languages (for example, an object code, an assembly language, or a high-level language such as C, C++, or JAVA). The computer instructions may be distributed in any form as a removable storage medium with accompanying printed or electronic documentation (for example, shrink wrapped software), preloaded on the greenhouse gas emission analysis system 32 (for example, on system ROM or fixed disk), or distributed from a server or electronic bulletin board over a communication system (for example, the Internet or World Wide Web). In addition, in certain embodiments, the processer may be part of a cloud-based computing environment, and the computer program logic may be executed by the edge device in the cloud-based computing environment.

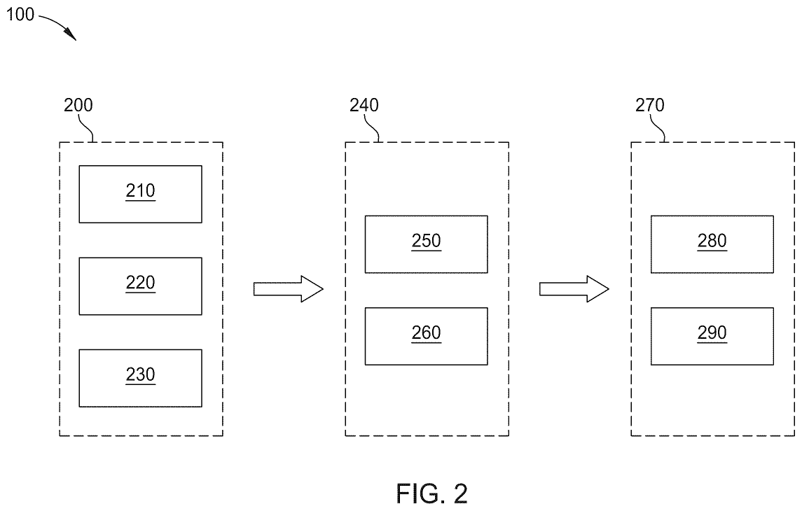

FIG. 2 is a block diagram of data planning design and gas flame emissions determination that may be used in system 100 of FIG. 1. The processor 150 may receive input data 200, such as the gas composition data 210, flow rate data 220, and combustion efficiency data 230, from a variety of date sources, such data as gas analyzer 130 and flow meter 140, among others. In some embodiments, the input data 200 may be transmitted to a location 240 having the processor 150 that is adjacent or remote from a worksite or monitored facility.

The input data 200 from various devices may be transmitted to the processor, and the processor includes mathematical models used to determine the gas emissions. For example, the processor 150 may include mathematical models 250 and a variety of interactive functions 260. The mathematical models 250 may be generated using various mathematical algorithms associated with data analysis, processing, and simulations related to the gas flare emissions and data correlation, such as correlating gas composition data 210, flow rate data 220, among others. The interactive functions 260 may provide functionalities to support the data analysis, processing, and calculations related to the gas flare emissions.

Based on data analysis and calculations from mathematical models 250, processor 150 may generate a variety of output data 270. For example, the output data 270 may include emission data 280 as well as outputted input data 290 of one or more sources of input data 200, such as the gas composition data 210.

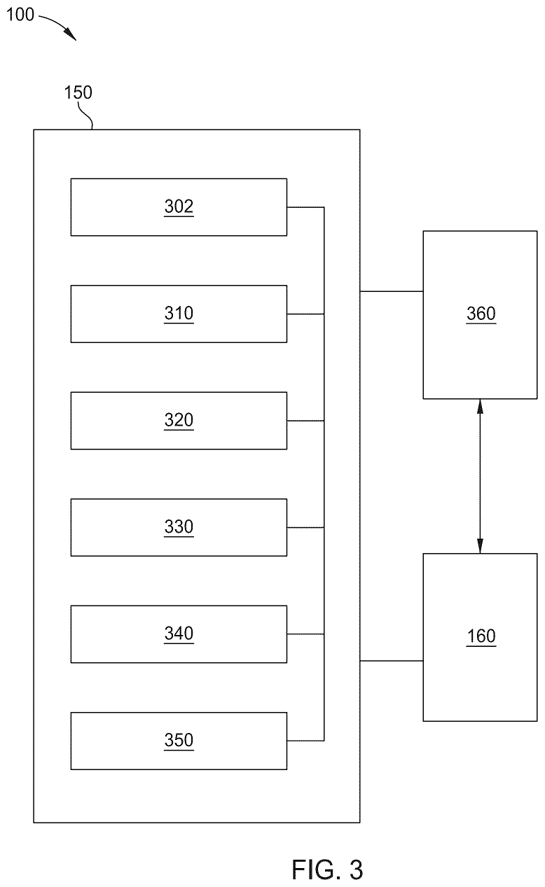

FIG. 3 is a block diagram of an example implementation of the example system 100 of FIG. 1 including the processor 150 of FIGS. 1-2. In FIG. 3, the emission system 100 includes the processor 150 to calculate gas flare emissions and process emission data to determine carbon equivalents of the emission gases. In FIG. 3, processor 150 includes the gas analyzer 130 data input 302, a flow rate meter 140 data input 310, emissions calculator 320 utilizing the mathematical models 250, an output generator 330, an gas flare efficiency input 340, and a database 350.

In FIG. 3, processor 150 includes the gas analyzer 130 data input 302 to continuously obtain and process gas concentration data. In some examples, the gas analyzer 130 processes samples of the gas feed 110 to determine the concentrations of gas components of the gas feed 110 for data input 302. For example, using various technologies described herein, the gas analyzer 130 can calculate a volume concentration of the gas feed components. For example, the gas analyzer 130 can calculate a volume concentration of components selected from the group consisting of methane, ethane, propane, isobutane, n-butane), isopentane, n-pentane, nitrogen gas (N₂) (Nitrogen), carbon dioxide, hydrogen sulfide, and combinations thereof. In some examples, the gas analyzer 130 obtains operating parameters and/or processing conditions, such temperature or pressure for data input 302.

In the illustrated example of FIG. 3, the processor 150 includes flow rate meter 140 data input 310. The flow rate meter 140 can obtain gas volumetric flow rate data or mass flow rate data for data input 310, and such data can be continuously provided and inputted into the processor. In some examples, the flow rate meter 140 data input 310 includes operating parameters and/or processing conditions, such temperature or pressure.

In the illustrated example of FIG. 3, the processor 150 includes the emissions calculator 320 to calculate emissions from the gas flare. In some examples, the emissions calculator 320 determines the emissions of the separate components and the carbon equivalents of the emissions. The emissions calculator 320 can process data in mathematical models based on mathematical formulas and chemical formulas. The mathematical models may be from either a commercial source, such as commercially available software or services, or proprietary sources. For example, the emissions calculator 320 may mathematically determine the amounts of carbon dioxide, carbon monoxide, unburned hydrocarbons CxHy, such as methane (CH₄), and soot, which may be in the form of smoke, and may further calculate the carbon dioxide equivalents of those components based on mathematical formulas and chemical formulas.

In one embodiment, the emissions calculator 320 processes the gas composition data, specifically the concentrations of hydrogen sulfide (H₂S), carbon dioxide (CO₂), nitrogen gas (N₂), methane (abbreviated as C₁), ethane (abbreviated as C₂), propane (abbreviated as C₃), isobutane (abbreviated as i-C₄), n-butane (abbreviated as n-C₄), isopentane (abbreviated as i-C₅), and n-pentane (abbreviated as n-C₅) to calculate total gas throughput. Using either the volumetric flow rate (in million standard cubic feet per day (MMSCFD)) or the mass flow rate (in kg/s) as inputs, the system applies a combustion efficiency of 98%, a typical value for flaring systems. The emissions calculator 320 in this embodiment, uses a linear mathematical regression model to estimate the resulting emissions produced during flaring. These emissions are quantified in terms of CO₂ (carbon dioxide), CH₄ (methane), NOx (nitrogen oxides), CO (carbon monoxide), SO₂ (sulfur dioxide), and soot. The model uses empirical relationships derived from combustion chemistry and validated datasets to predict emission outputs under the assumed combustion conditions.

In one embodiment, the data may be processed or calculated by a sequence as follows.

Initially, the gas volume flow rate meter 140 data input 310 is converted from MMSCFD to standard cubic meters per day using a conversion factor of 0.0283168 m³ per standard cubic feet (scf) to form the total gas volume.

Then the total moles of gas, molar flow, is calculated by dividing the total gas volume by standard molar volume (23.645 m³ per kmol), which gives the total amount of gas in kmol per day.

The molar flow of the respective components are calculated by multiplying the total molar flowrate by the mole fraction (or ppm/1,000,000) of the specific gas component from the gas feed 110 data input 302 to find the molar flowrate of the respective component(s).

Next, the combustion efficiency is applied, for example, the 98% combustion efficiency rate, thereby taking 98% of the component's molar flowrate, assuming near-complete combustion. The combustion efficiency is provide from the gas flare efficiency input 340.

Then the emission components' (or product) moles are determined by using the combustion reaction to determine how many moles of emission products are formed. For example, one mole of H₂S produces one mole of SO₂. This is performed for all gas feed components to emission components.

The emission components' (or product) moles are converted to mass by multiplying the molar flow of the emission component by the respective molecular weight to calculate mass in kilograms per day.

Then, the mass is converted into tonnes by dividing the mass in kilograms per day by 1,000 to result in metric tonnes per day. This information can then be displayed on the output devices among other uses.

In one example calculation following the process embodiment herein, the sulfur dioxide, SO₂, emissions are calculated as follows using the following data:
H₂S mole fraction in gas: 25%=0.25 mol fraction,
volumetric flowrate of gas: 1 MMSCFD,
molecular weight of H₂S: 34.08 g/mol,
molecular weight of SO₂: 64.07 g/mol,
combustion reaction (complete): 98% combustion efficiency,
chemical reaction formula, H₂S and 1.5 O₂ react to form SO₂ and H₂O, thereby 1 mol of H₂S produces 1 mol of SO₂.

Following one embodiment of the calculation sequence described herein:
1) Convert 1 MMSCFD to m³/day:

MMSCFD=28,316.8 m³/day,
2) Convert volume to kmol/day:

$$\frac{28 , 316.8 {m}^{3} / day}{23.645 {m}^{3} / k mol} = 1 , 197.4 k mol / day ,$$

3) Determine H₂S molar flow rate:

$$1 , 197.4 \frac{k mol}{day} * 0.25 = 299.35 \frac{k mol}{day} ,$$

4) Apply 98% Combustion Efficiency:

$$299.35 \frac{k mol}{day} * 0.98 = 293.36 \frac{k mol}{day} ,$$

5) Products moles H₂S to SO₂: 1:1 molar ratio:

$${SO}_{2} Produced = 293.36 \frac{k mol}{day} ,$$

6) Convert SO₂ produced to Mass (kg/day):

$$293.36 \frac{k mol}{day} * 64.06 \frac{kg}{k mol} = 18 , 790.1 kg / day ,$$

7) Convert to metric tonnes per day:

$$\frac{18 , 790.1 kg / day}{1000 \frac{kg}{tonnes}} = 18.79 \frac{metric tonnes}{day} of SO 2$$

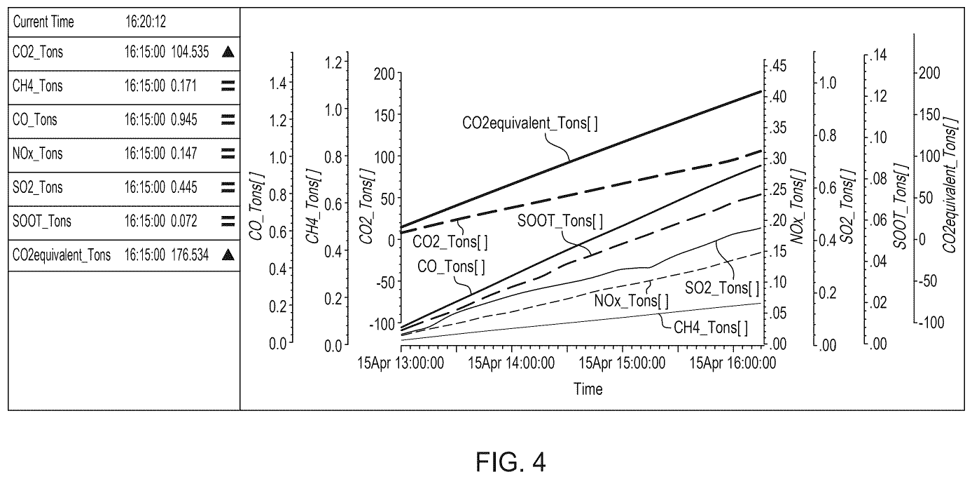

In the illustrated example of FIG. 3, the processor 150 includes the output generator 330 to generate a report or a log associated with an operation of the gas flare and output such data to one or more databases or display devices. In some examples, the output generator 330 generates a report including emissions data of each component of the emissions, such as mass calculations. As this calculated data is occurring continuously, such data can be provided both for specific times and gas concentrations and also used for total omissions over user determined periods of time. Such gas emissions output data can be provided continuously and can be presented in various formats, such as combined to calculate total emission over selected periods of time as shown in FIG. 4.

In some examples, the output generator 330 includes output data that can be provided to output devices 160, such as displayed on monitoring devices in a readable format by a user. An example of a format for a displayed gas emissions output is shown on FIG. 4. Examples of suitable output devices 160 include monitors, databases, data acquisition systems, and combinations thereof.

Referring to FIG. 4, FIG. 4 demonstrates that the gas composition being flared is continuously analyzed to calculate real-time emissions in metric tonnes as the gas flows to the flare. The real-time calculation enables informed operational decisions to minimize emissions by reducing flaring, set emission targets for specific activities, and monitor progress toward those targets effectively. For example, emissions of carbon dioxide (CO2_TONS), carbon monoxide (CO_TONS), methane (CH4) (CH4_TONS), and soot (SOOT_Tons) can all be calculated and displayed individually and plotted with the respective cumulative emissions over periods of time. The emissions calculator 320 may further calculate the carbon dioxide equivalents of those components based on mathematical formulas and chemical formulas and display such information continuously and/or cumulatively, such as illustrated by line (CO2equivalents_TONS) in FIG. 4. Other materials, such as nitrous oxide emissions (Nox_Tons) and sulfur dioxide emissions (SO2_TONS) can also be calculated and displayed.

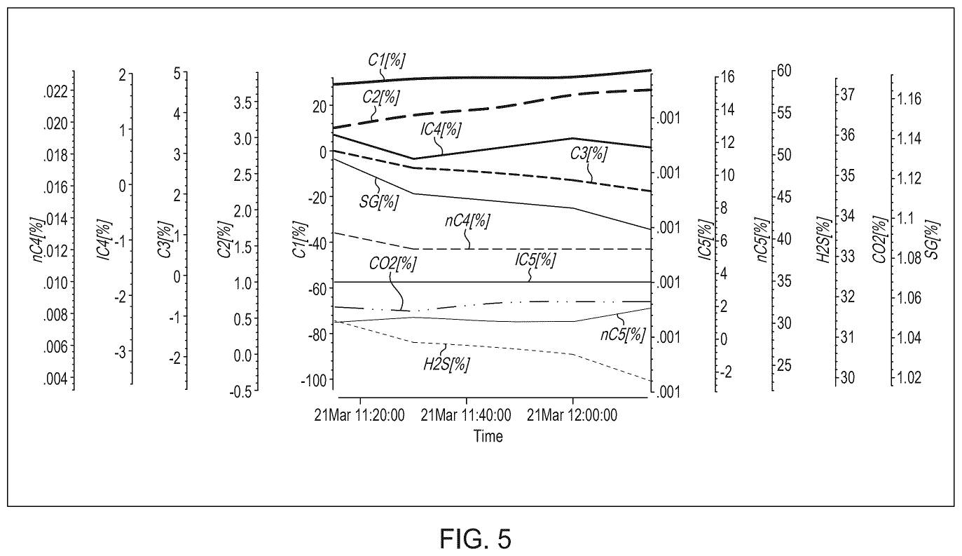

Similarly, as the processor receives input data, such input data can also be processed by the output generator 330 and displayed on monitoring devices in a readable format by a user. An example of such an output of gas concentration input data is shown in FIG. 5. Such output data can be provided continuously.

Referring to FIG. 5, FIG. 5 demonstrates that any device selected for use of gas analysis provides a real-time feed of gas composition in weight or molar mass percentages. These values are then used to calculate flared emissions based on flaring efficiency (factor), such as the standard 98% flaring efficiency factor. The flaring efficiency can vary depending on the flare technology used, and any efficiency factor can be applied to the composition data to determine emissions in metric tonnes.

As can be shown in FIG. 5, the various concentrations of components, such as by volume percents of the components can be shown. For example, FIG. 5 illustrates that the gas analyzer 130 can calculate a volume percent (concentration) of components selected from the group consisting of methane (C1 [%]), ethane (C2 [%]), propane (C3 [%]), isobutane (IC4 [%]), n-butane (nC1 [%]), isopentane (IC5 [%]), n-pentane (nC5 [%]), and hydrogen sulfide (H2S [%]), among other components, such as nitrogen gas (N2), and carbon dioxide, (not shown), can be shown continuously on the output device. As illustrated in FIG. 5, the output device allows a reader to see the fluctuations in the amounts of the various components as the feed gas is processed to the flare to improve understanding of the emission outputs.

Referring back to FIG. 3, the processor 150 includes the database 350 to record data, such as input data and calculated emissions data. The database 350 can be implemented by a volatile memory (for example, a Synchronous Dynamic Random Access Memory (SDRAM), Dynamic Random Access Memory (DRAM), RAMBUS Dynamic Random Access Memory (RDRAM), etc.) and/or a non-volatile memory (for example, flash memory). The database 350 can additionally or alternatively be implemented by one or more double data rate (DDR) memory, such as DDR, DDR2, DDR3, mobile DDR (mDDR), etc. The database 350 can additionally or alternatively be implemented by one or more mass storage devices such as hard disk drive(s), compact disk drive(s) digital versatile disk drive(s), etc. While in the illustrated example the database 350 is illustrated as a single database, the database 350 can be implemented by any number and/or type(s) of databases. Furthermore, the data stored in the database 350 can be in any data format such as, for example, binary data, comma delimited data, tab delimited data, structured query language (SQL) structures, etc.

In system 100 of FIG. 3, processor 150 and the system 100 are communicatively coupled to an example network 360. Network 360 of the illustrated example of FIG. 3 may be an intranet or the Internet. However, network 360 can be implemented using any suitable wired and/or wireless network(s) including, for example, one or more data buses, one or more Local Area Networks (LANs), one or more wireless LANs, one or more cellular networks, one or more private networks, one or more public networks, etc. In some examples, the network 360 enables the processor 150 to be in communication with the system components, such as the gas analyzer 130 and flow meter 140, and the processor 150 can obtain information from the system components via the network 360. In some examples, the network 360 enables the processor 150 to communicate with the database 350 or an external computing device, such as a server, to store the information obtained by the processor 150. In such examples, the network 360 enables the processor 150 to retrieve and/or otherwise obtain the stored information for processing. In some examples, the network 360 is a cloud-based network, which can perform cloud-based data storage, analytics, big data analysis, deep machine learning, among other processes, based on information obtained and/or processed by the processor 150. Processor 150 can be an Internet of Things (IoT) device enabled to facilitate capturing, communicating, analyzing, and acting on data generated by networked objects and machines.

While an example manner of implementing the system 100 and the processor 150 of FIGS. 1-3, one or more of the elements, processes, and/or devices illustrated in the figures may be combined, divided, re-arranged, omitted, eliminated, and/or implemented in any other way. Further, the example gas analyzer 302, the example flow rate meter 140 data input 310, the example emissions calculator 320, the example output generator 330, the example gas flare efficiency input 340, the example database 350, and/or, more generally, the example processor 150 of FIG. 3 may be implemented by hardware, software, firmware, and/or any combination of hardware, software, and/or firmware. Thus, for example, any of the example gas analyzer 302, the example flow rate meter 140 data input 310, the example emissions calculator 320, the example output generator 330, the example gas flare efficiency input 340, the example database 350, and/or, more generally, the example processor 150 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)).

When reading any of the apparatus or system claims of this patent to cover a purely software and/or firmware implementation, at least one of the example gas analyzer 302, the example flow rate meter 140 data input 310, the example emissions calculator 320, the example output generator 330, the example gas flare efficiency input 340, and/or the example database 350 is/are hereby expressly defined to include a non-transitory computer readable storage device or storage disk such as a memory, a digital versatile disk (DVD), a compact disk (CD), a Blu-ray disk, etc., including the software and/or firmware. Further still, the example processor 150 of FIGS. 1-3 may include one or more elements, processes, and/or devices in addition to, or instead of, those illustrated in the figures, and/or may include more than one or all the illustrated elements, processes, and devices. As used herein, the phrase “in communication,” including variations thereof, encompasses direct communication and/or indirect communication through one or more intermediary components, and does not require direct physical (for example, wired) communication and/or constant communication, but rather additionally includes selective communication at periodic intervals, scheduled intervals, aperiodic intervals, and/or one-time events.

### Example Aspects

Implementation examples are described in the following numbered clauses:

Aspect 1: A method for continuous determining of gas emissions, comprising: providing a gas feed to a gas flaring device; continuously analyzing the gas composition of the gas feed; providing gas composition data of the gas feed to a processor; providing a gas volumetric rate or a gas mass rate to the processor; providing a gas flaring device combustion efficiency to the processor; and determining, by the processor, the gas flaring device emissions.

Aspect 2: The method of Aspect 1, further comprising determining carbon dioxide equivalents of the gas flaring device emissions.

Aspect 3: The method of any combination of Aspects 1-2, further comprising continuously providing data of the gas flaring device emissions to a display device.

Aspect 4: The method of Aspect 3, further comprising continuously providing an ongoing summation of the gas flaring device emissions to a display device.

Aspect 5: The method of any combination of Aspects 1-4, further comprising determining the gas composition specific gravity.

Aspect 6: The method of any combination of Aspects 1-5, wherein the gas flaring device emissions comprise emissions of carbon dioxide, carbon monoxide, methane, nitrous oxide, sulfur dioxide, soot or combinations thereof.

Aspect 7: The method of any combination of Aspects 1-6, wherein the analyzing the gas composition is performed during flowback operations.

Aspect 8: The method of any combination of Aspects 1-7, wherein the analyzing the gas composition of a gas in a gas line coupled to a gas flaring device comprises analyzing the gas composition by a process selected from the group consisting of Raman spectroscopy, tunable filter spectroscopy, mass spectroscopy, gas chromatography, tunable diode laser absorption spectroscopy, Fourier transform infrared spectroscopy, cavity ring-down spectroscopy, and combinations thereof.

Aspect 9: The method of Aspect 8, wherein the analyzing the gas composition of a gas in a gas line coupled to a gas flaring device comprises generating gas composition data comprising percentages of components comprising methane, ethane, propane, isobutane, n-butane, isopentane, n-pentane, nitrogen gas, carbon dioxide, hydrogen sulfide, and combinations thereof.

Aspect 10: The method of any combination of Aspects 1-9, wherein the providing a gas volumetric rate or a gas mass rate to the processor comprises measuring gas volumetric rate or gas mass rate comprises using one or more gas metering devices selected from the group consisting of an orifice metering device, a Coriolis metering device, a diaphragm metering device, a rotary metering device, a turbine metering device, and combinations thereof.

Aspect 11: The method of any combination of Aspects 1-10, wherein the analyzing the gas composition of the continuous feed of the gas comprises analyzing the gas composition using an in-line gas analyzer or an in-situ gas analyzer in the gas line coupled to the gas flaring device.

Aspect 12: A method for determining gas emissions, comprising: providing a continuous feed of a gas to a gas flaring device; continuously analyzing the gas composition of the continuous feed of the gas; providing continuous data to processor, comprising: providing gas composition data to the processor; providing a gas volumetric rate or a gas mass rate to the processor; providing a gas flaring device combustion efficiency to the processor; and determining, by the processor, the gas flaring device emissions.

Aspect 13: The method of Aspect 12, wherein the method further comprises one or more continuous processes selected from the group consisting of: determining the carbon dioxide equivalents of the gas flaring device emissions, providing data of the gas flaring device emissions to a display device, determining the gas composition specific gravity, and combinations thereof.

Aspect 14: The method of any combination of Aspects 12-13, wherein the gas flaring device emissions comprise emissions of carbon dioxide, carbon monoxide, methane, nitrous oxide, sulfur dioxide, soot or combinations thereof.

Aspect 15: The method of any combination of Aspects 12-14, wherein the analyzing the gas composition is performed during flowback operations.

Aspect 16: The method of Aspect 12-15, wherein the analyzing the gas composition of a gas in a gas line coupled to a gas flaring device comprises generating gas composition data comprising percentages of components comprising methane, ethane, propane, isobutane, n-butane, isopentane, n-pentane, nitrogen gas, carbon dioxide, hydrogen sulfide, and combinations thereof.

Aspect 17: The method of any combination of Aspects 12-16, wherein the gas composition data comprises percentages of components selected from the group consisting of methane, ethane, propane, isobutane, n-butane, isopentane, n-pentane, nitrogen gas, carbon dioxide, hydrogen sulfide, and combinations thereof, and the gas flaring device emissions comprise emissions of carbon dioxide, carbon monoxide, methane, nitrous oxide, sulfur dioxide, soot or combinations thereof.

Aspect 18: The method of any combination of Aspects 12-17, wherein the providing a gas volumetric rate or a gas mass rate to the processor comprises measuring gas volumetric rate or gas mass rate comprises using one or more gas metering devices selected from the group consisting of an orifice metering device, a Coriolis metering device, a diaphragm metering device, a rotary metering device, a turbine metering device, and combinations thereof.

Aspect 19: The method of any combination of Aspects 12-18, wherein the analyzing the gas composition of the continuous feed of the gas comprises analyzing the gas composition in a gas line fluidly coupled to a gas flaring device.

Aspect 20: for continuous determining of gas emissions including the methods of Aspects 1-18.

Aspect 21: non-transitory computer readable storage medium comprising instructions which, when executed, cause an apparatus or system machine to perform a method in accordance with any of Aspects 1-18.

### ADDITIONAL CONSIDERATIONS

The preceding description is provided to enable any person skilled in the art to practice the various aspects described herein. The examples discussed herein are not limiting the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various actions may be added, omitted, or combined. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, structure and functionality in addition to, or other than, the various aspects of the disclosure set forth herein. It should be understood that any aspect of the disclosure disclosed herein may be embodied by one or more elements of a claim.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, for example, a combination of a DSP and a microprocessor, a plurality of microprocessors, one or more microprocessors in conjunction with a DSP core, a system on a chip (SoC), or any other such configuration.

When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements. Moreover, any use of “top,” “bottom,” “above,” “below,” other directional terms, and variations of these terms is made for convenience, but does not mandate any particular orientation of the components.

In addition, as used herein, the terms “continuous”, “continuously”, “real time”, “real-time”, or “substantially real time” may be used interchangeably and are intended to described operations that are performed without any human-perceivable interruption between operations, for example, a system that operates without interruption or cessation. For example, data relating to the systems described herein may be collected, transmitted, and/or used in computations in “substantially real time”, such that data readings, data transfers, and/or data processing steps may occur once every second, once every 0.1 second, once every 0.01 second, or even more frequent, during operations of the systems (for example, while the systems are operating). In addition, as used herein, the terms “automatic” and “automated” are intended to describe operations that are performed are caused to be performed, for example, by system for determining gas emissions and outputting such data without human intervention.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (for example, a-a, a-a-a, a-a-b, a-a-c, a-b-b, a-c-c, b-b, b-b-b, b-b-c, c-c, and c-c-c or any other ordering of a, b, and c).

As used herein, “a processor,” “processor,” or “one or more processors” generally refer to a single processor configured to perform one or multiple operations or multiple processors configured to collectively perform one or more operations. In the case of multiple processors, performance of one or more operations could be divided amongst different processors, though one processor may perform multiple operations, and multiple processors could collectively perform a single operation. Similarly, “a memory,” “at least one memory,” or “one or more memories” generally refer to a single memory configured to store data and/or instructions, or multiple memories configured to collectively store data and/or instructions.

As used herein, the term “determining” encompasses a wide variety of actions. For example, “determining” may include calculating, computing, processing, deriving, investigating, looking up (for example, looking up in a table, a database or another data structure), ascertaining and the like. Also, “determining” may include receiving (for example, receiving information), accessing (for example, accessing data in a memory) and the like. Also, “determining” may include resolving, selecting, choosing, establishing and the like.

The methods disclosed herein comprise one or more actions for achieving the methods. The method of actions may be interchanged with one another without departing from the scope of the claims. In other words, unless a specific order of actions is specified, the order and/or use of specific actions may be modified without departing from the scope of the claims. Further, the various operations of methods described above may be performed by any suitable means capable of performing the corresponding functions. The means may include various hardware and/or software component(s) and/or module(s), including, but not limited to a circuit, an ASIC, or processor.

The following claims are not intended to be limited to the aspects shown herein but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for continuous determining of gas emissions, comprising:
providing a gas feed to a gas flaring device;
continuously analyzing a gas composition of the gas feed with an analyzer;
providing gas composition data of the gas feed from the analyzer to a processor;
providing a gas volumetric rate or a gas mass rate to the processor;
providing a gas flaring device combustion efficiency to the processor; and
determining, by the processor, the gas flaring device emissions.

2. The method of claim 1, further comprising determining carbon dioxide equivalents of the gas flaring device emissions.

3. The method of claim 1, further comprising continuously providing data of the gas flaring device emissions to a display device.

4. The method of claim 3, further comprising continuously providing an ongoing summation of the gas flaring device emissions to a display device.

5. The method of claim 1, further comprising determining the gas composition specific gravity.

6. The method of claim 1, wherein the gas flaring device emissions comprise emissions of carbon dioxide, carbon monoxide, methane, nitrous oxide, sulfur dioxide, soot or combinations thereof.

7. The method of claim 1, wherein the analyzing the gas composition is performed during flowback operations.

8. The method of claim 1, wherein the analyzing the gas composition of a gas in a gas line coupled to a gas flaring device comprises analyzing the gas composition by a process selected from the group consisting of Raman spectroscopy, tunable filter spectroscopy, mass spectroscopy, gas chromatography, tunable diode laser absorption spectroscopy, Fourier transform infrared spectroscopy, cavity ring-down spectroscopy, and combinations thereof.

9. The method of claim 8, wherein the analyzing the gas composition of a gas in a gas line coupled to a gas flaring device comprises generating gas composition data of components comprising methane, ethane, propane, isobutane, n-butane, isopentane, n-pentane, nitrogen gas, carbon dioxide, hydrogen sulfide, or combinations thereof.

10. The method of claim 1, wherein the providing a gas volumetric rate or a gas mass rate to the processor comprises measuring gas volumetric rate or gas mass rate comprises using one or more gas metering devices selected from the group consisting of an orifice metering device, a Coriolis metering device, a diaphragm metering device, a rotary metering device, a turbine metering device, and combinations thereof.

11. The method of claim 1, wherein the analyzing the gas composition of the continuous feed of the gas comprises analyzing the gas composition using an in-line gas analyzer or an in-situ gas analyzer in the gas line coupled to the gas flaring device.

12. A method for determining gas emissions, comprising:
providing a continuous feed of a gas to a gas flaring device;
continuously analyzing the gas composition of the continuous feed of the gas;
providing continuous data to processor, comprising:
providing gas composition data to the processor;
providing a gas volumetric rate or a gas mass rate to the processor;
providing a gas flaring device combustion efficiency to the processor; and
determining, by the processor, the gas flaring device emissions.

13. The method of claim 12, wherein the method further comprises one or more continuous processes selected from the group consisting of:
determining the carbon dioxide equivalents of the gas flaring device emissions,
providing data of the gas flaring device emissions to a display device,
determining the gas composition specific gravity, and
combinations thereof.

14. The method of claim 12, wherein the gas flaring device emissions comprise emissions of carbon dioxide, carbon monoxide, methane, nitrous oxide, sulfur dioxide, soot, or combinations thereof.

15. The method of claim 12, wherein the continuously analyzing the gas composition is performed during flowback operations.

16. The method of claim 12, wherein the continuously analyzing the gas composition of the continuous feed of the gas comprises analyzing the gas composition by a process selected from the group consisting of Raman spectroscopy, tunable filter spectroscopy, mass spectroscopy, gas chromatography, tunable diode laser absorption spectroscopy, Fourier transform infrared spectroscopy, cavity ring-down spectroscopy, and combinations thereof.

17. The method of claim 12, wherein the gas composition data comprises components selected from the group consisting of methane, ethane, propane, isobutane, n-butane, isopentane, n-pentane, nitrogen gas, carbon dioxide, hydrogen sulfide, and combinations thereof, and the gas flaring device emissions comprise emissions of carbon dioxide, carbon monoxide, methane, nitrous oxide, sulfur dioxide, soot or combinations thereof.

18. The method of claim 12, wherein the providing a gas volumetric rate or a gas mass rate to the processor comprises measuring gas volumetric rate or gas mass rate comprises using one or more gas metering devices selected from the group consisting of an orifice metering device, a Coriolis metering device, a diaphragm metering device, a rotary metering device, a turbine metering device, and combinations thereof.

19. The method of claim 12, wherein the analyzing the gas composition of the continuous feed of the gas comprises analyzing the gas composition in a gas line fluidly coupled to a gas flaring device.

20. A system for continuous determining of gas emissions, comprising:
a gas flaring device;
a gas feed line fluidly coupled to the gas flaring device;
a continuous gas analyzer coupled to the gas feed line;
a processor in electronic communication with the continuous gas analyzer, wherein the processor is adapted to receive:
gas composition data from the continuous gas analyzer,
gas volumetric rate or gas mass rate data, and
gas flaring device combustion efficiency data,
and to output emission data; and
a display device adapted to receive continuous output emission data from the processor.
